(19) United States
(12) Patent Application Publication
(10) Pub. No.: US 2022/0233086 A1
(43) Pub. Date: Jul. 28, 2022

(54) DEVICE AND METHOD FOR DETECTING A PRESSURE IN A RUMEN OF A RUMINANT

(71) Applicant: DROPNOSTIX GMBH, Potsdam (DE)

(72) Inventor: Michael BREITENSTEIN, Potsdam (DE)

(73) Assignee: DROPNOSTIX GMBH, Potsdam (DE)

(21) Appl. No.: 17/611,307

(22) PCT Filed: May 15, 2020
(86) PCT No.: PCT/EP2020/063594

§ 371 Date: Nov. 15, 2021

(30) Foreign Application Priority Data
May 16, 2019 (EP) 19174882.1

(51) Int. Cl. A61B 5/03; A61B 5/07; A61B 5/00

(52) U.S. Cl. CPC A61B 5/036; A61B 5/073; A61B 2503/40; A61B 2562/0247; A61B 5/6871

(57) ABSTRACT

The invention relates to a device (1) for detecting a pressure in a mammal's stomach, particularly in a rumen of a ruminant, the device (1) comprising at least the following components:
a sensing volume (12),
a pressure sensor (9) configured and arranged to detect a gas pressure in the sensing volume (12),
an elastic membrane (5) forming a deformable wall portion of the sensing volume (12), wherein the elastic membrane (5) is configured and arranged to deform in response to a pressure difference over the elastic membrane (5), wherein the elastic membrane (5) comprises a gas-permeable portion allowing a pressure difference over the elastic membrane (5) to equalize.

The invention relates to a device and a method for detecting a gastric pressure of a mammal, particularly a pressure in the reticulorumen of a ruminant, more particularly the pressure in the rumen of cattle.

In the absence of technical aids, diseases of ruminants associated with modern agriculture, such as for example subacute ruminal acidosis (SARA), are for the most part only possible to detect subsequently or indirectly.

SARA in particular is a serious agricultural problem in both health and economic terms. SARA may for instance be a trigger for reduced food intake, reduced milk yield, diarrheal diseases, inflammation of the ruminal mucosa, hoof diseases (laminitis, bleeding from the sole and sole ulcers) and lameness.

A possibility for detecting SARA involves measuring the activity of the rumen. SARA is known to be accompanied by reduced rumen movement.

In general, livestock producers face a need to reliably identify in each animal the physiological condition of the animal.

DE 299 11 803 U1 describes a device for determining gastric motility which detects the movement of the stomach with a piezoelectric sensor element. This, however, has the disadvantage that merely bending the sensor element generates evaluable signals. In addition, pressure changes in the organ are not detected if they do not bring about any deformation of the sensor element.

US 2008/0236500 A1 teaches a sensor device that is designed to permanently remain in the rumen of the ruminant. The sensor device comprises a pressure sensor for sensing a gas pressure in a gas tight sensing volume covered by a sealing diaphragm.

However, long-term or slow pressure changes in the rumen might lead to a reduced long-term stability (change of pressure baseline) and pressure resolution (saturation or non-ideal pressure range of the sensor) of the sensor data.

An object of the present invention is to provide a device for detecting a pressure in a mammal's stomach, particularly in a reticulorumen of a ruminant that is insensitive to slow or long-term pressure changes, while maintaining pressure sensitivity on short time scales. The object is achieved by the device having the features of claim 1.

Advantageous embodiments are described in the subclaims.

According to claim 1 the devices comprises at least the following components:
a closed gas-filled sensing volume,
a pressure sensor configured and arranged to detect a gas pressure, particularly a gas pressure change in the sensing volume,
an elastic membrane forming a deformable wall portion of the sensing volume, wherein the membrane is configured and arranged to deform in response to a pressure difference over the elastic membrane.

The invention is characterized in that the elastic membrane comprises at least one gas-permeable portion allowing a pressure difference, in the range encountered in rumens of ruminants, over the elastic membrane to equalize, such as to render the device insensitive to slow pressure changes.

The gas-permeability of the gas-permeable portion can be either a selective permeability that allows only certain gases to pass the gas-permeable portion or a non-selective portion that allows essentially any gas in the rumen of the ruminant to pass the membrane.

Particularly, the pressure sensor is configured and arranged to detect an atmospheric pressure, particularly only an atmospheric pressure, in the sensing volume, i.e. the pressure sensor is not sensitive to a selected gas but to the total gas pressure. The terms “atmospheric gas pressure” and “atmospheric pressure” particularly refer to the total gas pressure evoked by all gases in the gas-filled sensing volume, i.e. it is not a gas-selective pressure, or a partial pressure.

The terms “atmospheric gas pressure” and “atmospheric pressure” are used interchangeably in the current specification and have the same meaning.

In contrast to partial pressures—that strictly spoken are measures of concentrations only—the determination of the atmospheric pressure allows for a reliable determination of the pressure independent of any other chemical processes in the rumen that might affect the partial pressure of a specific gas, such a CO₂ or NO.

Particularly, the elastic membrane forms liquid-tight deformable wall portion of the sensing volume, wherein the elastic membrane covers an opening of the sensing volume and is configured and arranged to deform over the opening in response to a pressure difference over the elastic membrane, such that an atmospheric pressure change in the rumen is instantaneously detectable in the sensing volume.

According to this feature, the device is designed to provide the response times required for resolving rumen activity of the ruminant. In contrast, device for partial gas pressure measurements are not configured to resolve rumen activities in form of pressure changes, as the response time of said devices is too slow.

In order to provide a long-term application to the ruminant, it is necessary for the device to provide a means for pressure equalization between the sensing volume and the rumen of the animal for omitting measurement artefacts caused by a gas pressure build up inside the sensing volume that might be caused by the device itself and that is not related to ruminal activities.

The device according to the invention solves this problem.

For this reason, the elastic membrane might comprise a gas-permeable portion allowing a pressure difference over the elastic membrane to equalize within a predefined time interval, wherein said time interval is in the range of one second to 1.200 seconds or 300 seconds.

Particularly, said time interval is in the range of 10 seconds and 150 seconds, more particularly within the range of 30 seconds and 100 seconds.

With the pressure equalizing over the membrane, the membrane returns in its particularly planar non-deformed, equilibrium configuration.

These time intervals allow for a precise estimation of the pressure changes in the rumen of the animal while omitting any significant artificial pressure build up in side the sensing volume.

In order to arrive at the time intervals and to provide sufficient stability, while maintaining elasticity, the membrane, particularly the gas-permeable portion has a thickness between 100 μm and 700 μm. Membranes in this thickness regime might not be produced by means of extrusion processes, but for example by milling processes.

According to another embodiment of the invention, the membrane or the gas permeable membrane portion is made by a milling process.

For operation, the device is applied to the ruminant and is configured to remain in the rumen of the ruminant, where the device measures the pressure.

Contractions of the rumen are typically in the range of several seconds, e.g. in the range between 2 to 6 seconds. The device is configured to detect these contractions by means of a corresponding pressure change in the rumen, such that an activity of the rumen can be determined.

The device is particularly configured to be insensitive to pressure changes happening on a slower time scale than these contractions.

According to an embodiment of the invention, the device has particularly a bolus or cylinder shape, i.e. the device is of elongate configuration (in an axial direction) and, in the axial direction, has two free mutually opposing ends, wherein for example at one of such end, the elastic membrane covering the sensing volume with the pressure sensor may be provided.

Said ends are particularly rounded having not protruding components or portions (to reduce any risk of injury).

The device is particularly configured to permanently remain in the stomach of the livestock animal when it has been administered to the livestock animal and in particular is not excreted by rumination or digestion.

In this context, the term “permanent” means in particular that the device can only be removed from the stomach of the livestock animal after death or by surgery.

Ruminants have a plurality of stomachs which digest their diet. In a ruminant, the stomach in this connection in particular, but not necessarily exclusively, denotes the ruminant's rumen.

The device is advantageously usable in ruminants, in particular in cattle, in particular in dairy cows.

The device may be introduced into the livestock animal's stomach for example via its gullet. In the stomach, it can detect gastric pressure and the movements of the stomach, i.e. gastric motility, in order to obtain measured values for the intended period of operation, a minimum of 60 days being of relevance. Particularly, the device may be configured for several years of operation in the animal's stomach without interruption.

According to another embodiment of the invention, the device is configured and adapted to record the pressure in the sensing volume with a time resolution better than 10 seconds, particularly better than 5 seconds, more particularly equal to or better than 1 second, wherein a pressure change over the elastic membrane is transmitted to the sensing volume instantaneously.

Time resolution of the device plays a crucial role in order to be capable to detect ruminal contractions.

Time resolution can be adjusted by adjusting the thickness of the membrane.

The pressure sensor is configured to detect pressure and pressure changes in the sensing volume by means of suitable, for example piezoelectric, resistive, capacitive, I or magnetic, pressure sensors or even a micro-electromechanical systems (MEMS).

According to another embodiment of the invention, the pressure sensor is a piezo-resistive pressure sensor, particularly a digital piezo-resistive pressure sensor, configured to detect an atmospheric gas pressure in the sensing volume, particularly wherein the pressure sensor has an absolute pressure sensitivity in the range of 0.25 hPa and 3.0 hPa, particularly wherein the sensor has a relative pressure sensitivity in the range of 0.05 hPa and 0.5 hPa, more particularly wherein the pressure sensor has a sampling rate in the range of 10 Hz to 0.2 Hz, particularly in the range of 5 Hz and 0.5 Hz.

A piezo-restive sensor is particularly robust and energy-preserving, while providing the necessary specifications for detecting pressure changes in the rumen.

Particularly, the pressure sensitivity range and the sampling rate are chosen such that the comparably fast contractions (a few seconds) of the rumen can be recorded and resolved by the pressure sensor.

The measurement data from the pressure sensor may then be evaluated, for example by means of an electrical circuit, and forwarded to a microcontroller in which further processing steps take place.

The electric circuit and/or the microcontroller can be comprised in the device or be externally arranged.

The stability of the device and particularly a casing of the device in mechanical terms and in terms of its specific environment can be achieved by including for example POM (polyoxymethylene), PVC (polyvinyl chloride) or PEEK (polyetherether ketone) or sheathing the casing with these or other biocompatible polymers.

It is noted, that the elastic membrane is particularly not covered by additional components or protective layers from the outside.

The device is for example in the form of a pill.

The sensing volume is gas-tight except of the gas-permeable portion, i.e. gas can only enter or exit the sensing volume through the gas-permeable portion of the membrane.

According to another embodiment of the invention, a gas permeability of the gas-permeable portion is adjusted such that a gas pressure difference over the elastic membrane is sustained longer than an equalizing time of one second, particular ten seconds, more particularly 30 seconds.

As any pressure difference over the membrane starts equalizing immediately due to the gas-permeability of the gas-permeable portion, the expression “sustaining the pressure difference over the elastic membrane longer than an equalizing time” has to be understood for example in the sense that the pressure difference over the membrane has not completely decayed after said equalizing time.

This embodiment allows for reliable detection of pressure changes by the device, as there is sufficient time to detect the pressure change qualitatively and particularly quantitatively as well. The pressure sensor is particularly configured accordingly such as to have a high enough temporal resolution to detect such pressure changes.

According to one embodiment of the invention, the pressure difference has decayed not more than 50% at said equalizing time or during the time interval.

According to one embodiment of the invention, the pressure difference has decayed more than 95% within 30 minutes.

The gas-permeability of the membrane can for example be adjusted by adjusting the thickness of the gas-permeable portion.

According to another embodiment of the invention the gas permeability of the gas-permeable portion is adjusted such that a gas pressure difference over the elastic membrane is sustained shorter than 30 minutes.

Also in this embodiment the definitions and explanations from the previous embodiments apply with regard to the immediate start of pressure equalization.

This embodiment allows for sufficient time to render the device insensitive to slow changing or long-term pressure variations.

According to another embodiment of the invention, the elastic membrane consists of the gas-permeable portion.

According to this embodiment the whole elastic membrane is gas-permeable. This embodiment is particularly cost-efficient as only a single type of membrane needs to be manufactured.

According to another embodiment of the invention, the sensing volume is enclosed liquid tight by the device, wherein the elastic membrane and particularly the gas-permeable portion is liquid tight.

According to another embodiment of the invention, the gas-permeable portion, particularly the whole membrane, has a microscopic, particularly a molecular structure that provides the gas-permeability to the gas-permeable portion/membrane, particularly wherein any channel or opening through the gas-permeable portion of the membrane, has only microscopic dimensions.

This embodiment allows for a long enough equalizing time for a pressure difference over the membrane to vanish.

Moreover, this embodiment allows for a liquid-tight membrane.

According to another embodiment of the invention, only the microscopic, particularly molecular structure provides the gas-permeability to the gas-permeable portion or the whole membrane.

According to another embodiment of the invention, the gas-permeable portion; particularly the whole membrane, comprises Polytetrafluoroethylene (PTFE). According to another embodiment of the invention, the whole elastic membrane, particularly the gas-permeable portion of the elastic membrane comprises or consists of a support structure, such as a mesh, gauze, or an elastic fabric, particularly wherein the support structure is coated, laminated with or consists of Polytetrafluoroethylene (PTFE), more particularly wherein the support structure is a polyester or polypropylene fleece.

According to another embodiment of the invention, the gas-permeable portion comprises Polytetrafluoroethylene (PTFE).

According to another embodiment of the invention, the gas-permeable portion, particularly the whole membrane consists of Polytetrafluoroethylene (PTFE).

PTFE is particularly suitable, as on the one hand it provides adjustable gas-permeability, and on the other hand it provides chemical inert properties resulting in a long-term stability of the device in a rumen. Moreover, PTFE can be produced as an elastic membrane in various thicknesses allowing to adjust the gas-permeability of the gas-permeable portion, such as to match the desired equalizing time.

PTFE is hydrophobic such that no liquid passes through the gas-permeable portion, keeping the sensing volume liquid tight. Growth of microorganisms is greatly reduced on PTFE, particularly due to its hydrophobic properties.

Furthermore, the gas-permeable portion of the membrane exhibits a strongly reduced tendency to clog or to accumulate dirt on its surface, adding to the long-term stability of the device.

A membrane portion comprising or consisting of PTFE particularly provides the required time interval for equalizing the gas-pressure in the sensing volume and the required gas-permeability of the membrane portion, particularly the suitable amount of gas-permeability of the gas-permeable portion of the membrane, when the thickness of the membrane is chosen according to the invention.

According to another embodiment of the invention, the gas-permeable portion has a thickness that is larger than 100 μm.

This embodiment provides a sufficiently resilient membrane, while having a comparably short equalizing time for pressure differences over the membrane.

According to another embodiment of the invention, the membrane thickness is larger than 300 μm and/or smaller than 500 μm, particularly wherein the thickness is 400 μm. This embodiment provides a sufficiently elastic membrane, while still having an acceptable equalizing time for pressure differences over the membrane.

According to another embodiment of the invention, the device comprises a rigid casing, wherein said casing comprises the pressure sensor and wherein the elastic membrane is attached to the casing such as to form the sensing volume.

The casing, or optionally bolus for short, can be encapsulated with a polymer (for example epoxy resin) and is preferably watertight.

According to another embodiment of the invention, the device has an average density and a volume, adjusted such that that the device permanently remains in a reticulorumen of a ruminant, particularly wherein the average density is larger than 2.0 g/cm³, particularly wherein the volume is larger than 50 cm³.

In another embodiment of the invention, the device has an average density less than 2.6 g/cm³, particularly wherein the average density is within the range of 2.2 g/cm³ and 2.4 g/cm³, particularly wherein the density is 2.3 g/cm³.

According to another one embodiment of the invention, the device has a volume between 50 cm³ and 250 cm³, particularly between 70 cm³ and 160 cm³, wherein the volume particularly preferably amounts to 120 cm³. These size ranges advantageously ensure the tolerability of the device in the stomach of the livestock animal, in particular of a cow.

According to another embodiment of the invention, the device comprises a radio wave transmission module configured to wireless transmit sensor data generated by the pressure sensor and/or other sensors, such as a temperature, and/or an acceleration sensor, wherein the transmission module is particularly arranged in the casing of the device.

The transmission module allows sending the pressure data to an external device such as a computer, a computerized system, which is set up outside the animal in order to evaluate and process the pressure data.

Typical frequency bands and transmission strengths which are non-hazardous to the livestock animal are used for this purpose.

According to another embodiment of the invention, the device comprises an energy source, particularly a battery for providing the components, such as the pressure sensor, the transmission module, the electronic circuit and/or the microprocessor of the device with electric energy. As the device can also comprise additional sensors such as other sensors like temperature or acceleration and motion sensors, the energy source is further configured to provide these additional components with energy as well.

The energy source and energy management of the device according to the invention are in particular designed such that active operation of the device, i.e. for example intermittent or periodic measurements by the pressure sensor and/or intermittent or periodic transmissions by the transmission module, is possible for a period of at least one year.

According to another embodiment of the invention, the device does not comprise a valve configured to adjust a gas pressure in the sensing volume.

This embodiment allows for a cost-efficient and less complex device.

According to another embodiment of the invention, the membrane covers an area of more than 0.5 cm², particularly more than 1 cm², particularly wherein the opening of the sensing volume covered by the membrane has an area of more than 0.5 cm², particularly more than 1 cm², particularly wherein the opening has an area of less than 10 cm², particularly wherein the opening has an elliptic, oval or circular shape.

According to another embodiment of the invention, the membrane has a diameter between 0.5 cm and 6 cm, particularly between 2 cm and 4 cm, particularly wherein the membrane is circular. Said diameter is particularly measured over the opening of the sensing volume.

According to another embodiment of the invention, the membrane extends planar over the sensing volume in the absence of a pressure difference over the membrane.

The membrane has particularly a circular shape which is for example located at one end of the device and covers the sensing volume that is for example enclosed on the remaining sides by the casing and the pressure sensor.

This embodiment using a planar membrane allows a minimal exposure of the membrane to the rumen such that the lifespan and robustness of the device is increased.

Producing non-planar membranes, particularly non-planar membranes comprising PTFE is almost impossible by common extrusion methods. Therefore, when coating a gas-sensor device with a PTFE film or membrane with the know extrusion processes, the coating cannot be performed such that larger openings in the range of 0.5 cm² or larger could be covered with a planar membrane portion of a PTFE-comprising membrane.

Therefore according to another embodiment of the invention, the membrane, particularly the PTFE membrane portion of the membrane is made using a milling process.

In a further embodiment of the invention, the device has a means that is configured to connect the membrane or the sensing volume detachably to the device, such that the sensing volume or the membrane is replaceable if, for example, the device is to be used in a further animal. A sensing volume/membrane which may have suffered severe wear can accordingly be exchanged comparatively simply without having to replace the complete device.

Such means may for example be embodied by a screw or clip connection. A corresponding closure means for the membrane is preferably of annular construction. For this purpose the device or the casing can be comprise two releasable arranged portions.

The problem according to the invention is also solved by a method for measuring a mammal's stomach pressure, particularly a pressure in a rumen of a ruminant, with a device according to the invention, the method comprising the following steps of:
Applying the device to the mammal's stomach, particularly the rumen of the ruminant,
Periodically or continuously detecting a pressure in the sensing volume, wherein the pressure is sampled with a time resolution better than one second, particularly only when a pressure difference over the membrane is detected,
Storing the detected pressure and/or the detected pressure change on a data storage of the device and/or particularly periodically or intermittently transmitting the detected pressure or pressure change to a receiver arranged on an outside of the mammal.

Particularly, the periodic or continuous detection of the pressure, particularly the atmospheric pressure, more particularly only the atmospheric pressure, in the sensing volume is sampled with a time resolution better than ten, particularly better than one second, particularly only when a pressure difference over the elastic membrane is detected, wherein a time resolution of the device for detecting a pressure change is better than or equal to 1 second, particularly wherein a pressure change is detected instantaneous.

The method allows for a precise and long-term monitoring of the rumen activity of ruminants, that could not be achieved with other methods before.

According to another embodiment of the method, the method further comprises the steps of:
From the detected pressure and/or the detected pressure change determining rumen contractions of the ruminant on a computer or the microprocessor,
Determining a rumen activity, particularly a rumen contraction frequency, from the rumen contractions on a computer or the microprocessor,
Providing the determined rumen activity to a person, such as the owner or proprietor or medical staff by means of a display.

Particularly, exemplary embodiments are described below in conjunction with the Figures. The Figures are appended to the claims and are accompanied by text explaining individual features of the shown embodiments and aspects of the present invention. Each individual feature shown in the Figures and/or mentioned in said text of the Figures may be incorporated (also in an isolated fashion) into a claim relating to the device according to the present invention.

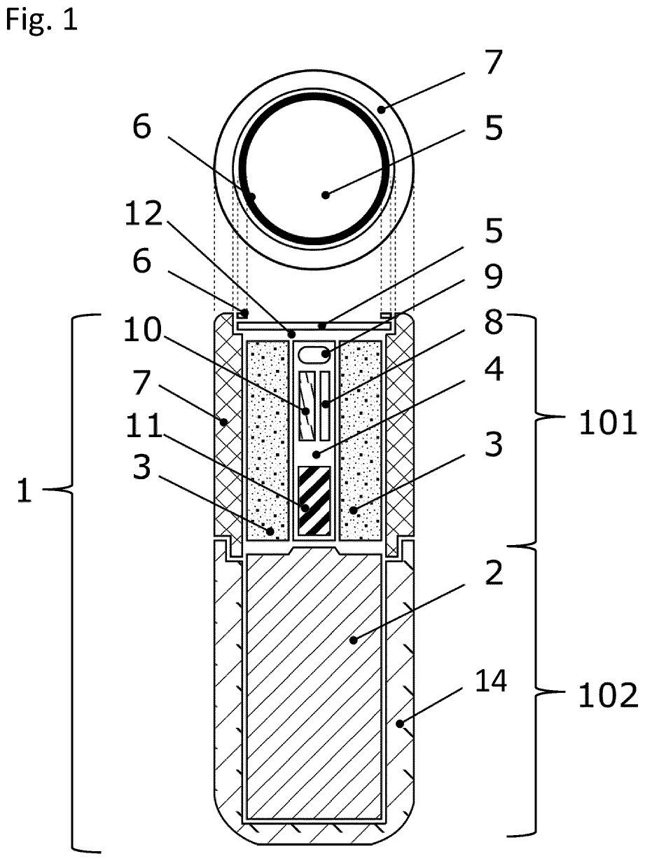

FIG. 1 shows two views of the device according to the invention. In the upper part of the FIG. 1 a top view of the device 1 is shown, wherein in the lower part of FIG. 1 a cross-sectional view is depicted. The device 1 has a bolus form, also optionally denoted bolus for short. The device 1 is subdivided into a first region or portion 101 and second region or portion 102. The first region 101 has a cylindrical casing 7 of POM. The second region 102 has a cylindrical casing 15 that is releasable attachable to the casing 7 of the first portion. The casing 7 comprises a packing piece 3, in which the electronics 4 of the device 1 are arranged. Selection of the polymer ensures on the one hand radio wave transmissibility and simultaneously on the other hand biocompatibility of the material.

The electronics 4 in this example comprise inter alia a transmission module 8, a pressure sensor 9 and a microcontroller 10 and a data storage 11. The pressure-sensitive part of the pressure sensor is accommodated in a sensing volume 12 (or can communicate therewith), the wall of which is at least in part likewise configured from the casing 7 of the first region 101 and the top of which is formed by an elastic, gas-permeable PTFE-membrane 5, i.e. a membrane that consists of PTFE, i.e. the whole membrane consists of a gas-permeable portion. The PTFE-membrane 5 has a thickness of 400 μm. The sensing volume 12 is located at one end of the first region 101 of the device 1 and is preferably arranged on the end face of the elongate device 1.

Deformation of the elastic PTFE-membrane 5 as a result of application of force brings about a change in pressure in the sensing volume 12 which can be detected by the pressure sensor 9. The sensing volume 12 is closed with a closure means in the form of a ring 6 which fixes the membrane 5 in place. The electronics 4 are internally interconnected in such a way that the measurement data from the pressure sensor 9 can be processed by the microcontroller 10, stored on the data storage 11 and sent by the transmission module 8 to an external computer. The measurement data can furthermore also be processed by the electronics 4 itself.

The second region 102 of the device 1 adjoins below the first region 101. The second region 102 has a cylindrical casing 14 (for example of a stainless steel) which comprises an energy source in the form of a battery 2 for supplying the electronics 4 with electricity.

The electronics 4 are for example activated by means of a latching circuit which is triggered with a reed contact. To this end, a magnet closes the reed contact from outside and so activates the electronics 4.

The electronics 4 can turn back off as a result of an appropriate level on reset of the latching circuit, so enabling a test run of the entire device 1.

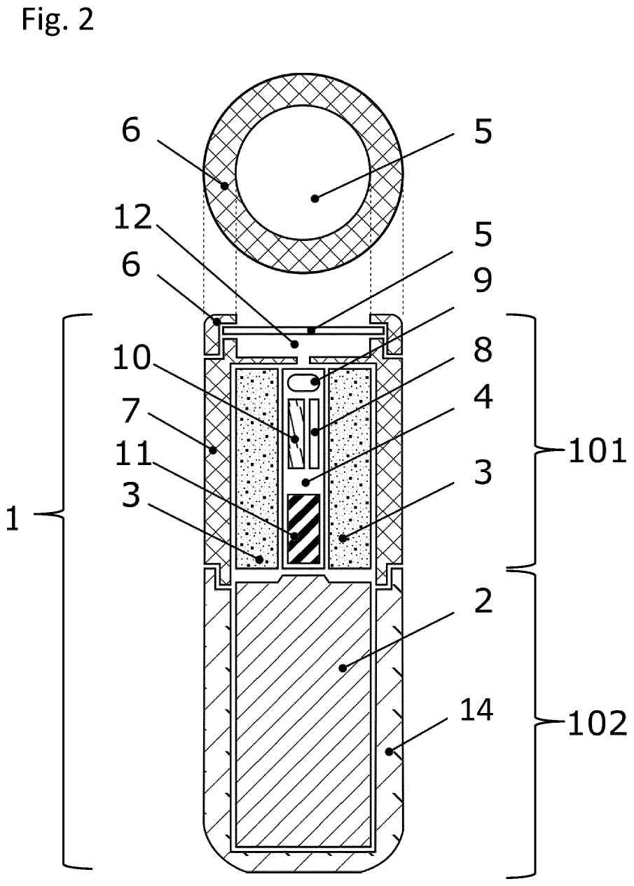

FIG. 2 shows a further configuration of the device 1. In the upper part of the FIG. 2 a top view of the device 1 is shown, wherein in the lower part of FIG. 2 a cross-sectional view is depicted. FIG. 2 shows the casing 14 of the second portion 102 of the device 1, which protectively encloses the energy source 2, the packing piece or alternatively weighting body 3, the electronics 4, the elastic gas-permeable PTFE-membrane 5, together with the sealing cap 6, the casing 7 of the first portion 101, the transmission module 8, the pressure sensor 9, as well as the microcontroller 10 and the data storage 11, and the sensing volume 12.

In contrast to the example shown in FIG. 1, the closure means 6 is configured as an annular sealing cap 6 which is configured to be screwed together with the device 1, in particular the casing 7, wherein the PTFE-membrane 5 is fixed in place over the sensing volume 12. To this end, the closure means 6, when screwed together as intended with the device 1 or casing 7, presses the PTFE-membrane 5 against a circumferential contact surface of the casing 7, wherein the PTFE-membrane 5 is fixed to the device 1 or the casing 7.

In particular, in an embodiment with a circumferential region, said annular sealing cap 6 presses against a circumferential edge region of the in particular circular PTFE-membrane 5. The PTFE-membrane 5 does not project beyond the sealing cap 6 in the axial direction of the device 1, as a result of which the PTFE-membrane 5 is provided with additional protection.

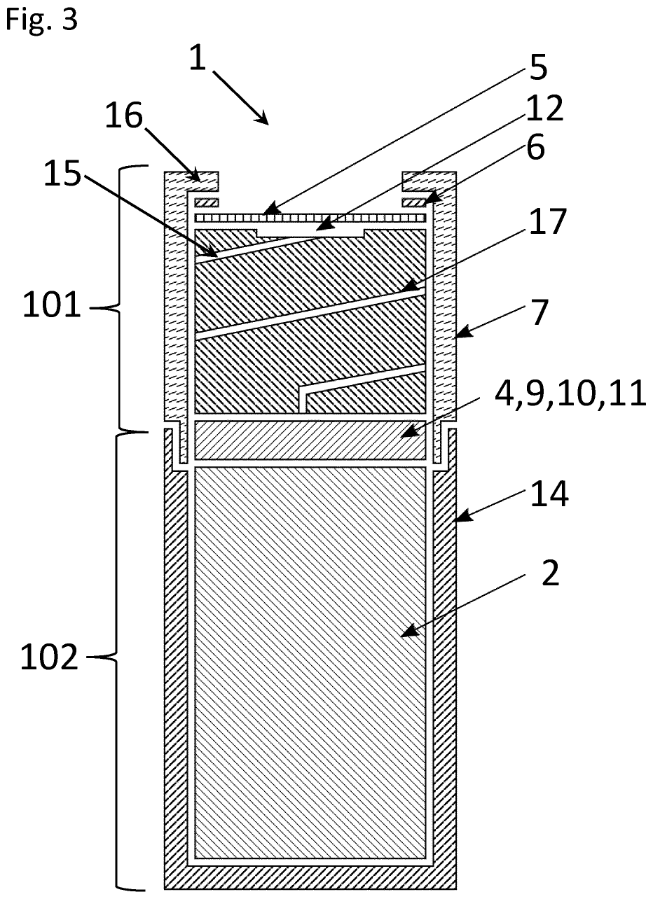

FIG. 3 shows a schematic and cross-sectional view of embodiment of the invention. The device 1 comprises a first portion 101 and a second portion 101 that are attached to each other along an axial direction of the device 1 and form a protective enclosure of the cylindrically-shaped device 1.

The first portion 101 comprises a cylindrical casing 7 forming a wall portion of the device 1, wherein at an upper opening, the device 1 is limited by an elastic, gas-permeable membrane 5 consisting of PTFE. The membrane 5 covers a gas-filled sensing volume 12 that is enclosed by the casing 7 and the membrane 5. The sensing volume 12 is fluidically connected by means of at least one channel (not shown) in the device 1 to a pressure sensor component 9. The pressure sensor component comprises the pressure sensor 9, a transmission module 4 for sending measured sensor data and for receiving external control data from an external device. Moreover, the pressure sensor component 9 comprises a microcontroller 10, as well as a data storage 11 for storing measured sensor data and received control data.

The device 1 comprises a mounting body 17 for an antenna and an antenna (not shown) that is arranged in specific channels 15 in the first portion 101 of the device 1 enclosed by the casing 7. The casing 7 is made from a radio-wave transparent material, such that transmission from and to the antenna of the device 1 is possible.

The second portion 102 comprises a casing 14 enclosing the energy source, namely the battery 2. The casing 14 is made from stainless steel in order to provide an inert but robust and stable casing for the second portion 102.

The membrane 5 is attached and fixed to the device by means of a membrane sealing ring 6 that is arranged circumferentially around an outer perimeter of the membrane 5. At a top portion of the casing 7, the casing 7 has a radially inward pointing protrusion 16 that is configured to protect and hold the sealing ring 6 onto the membrane 5.

The device shown in FIG. 3 is based on the same working principle as the devices shown in FIG. 1 and FIG. 2.

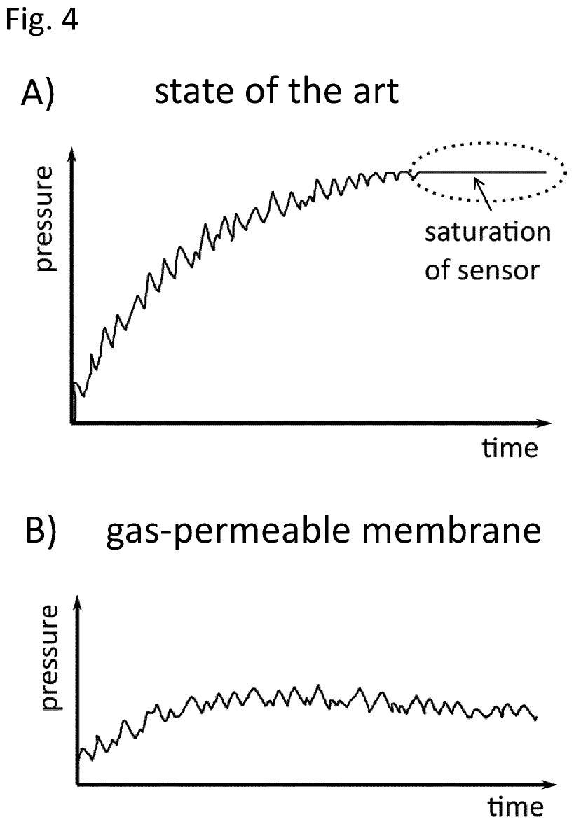

In FIG. 4 a schematic comparison of pressure measurements of conventional device for measuring ruminal activity to pressure measurement performed with a device according to the invention. Conventional device feature non-gas-permeable membranes. In both graphs the rumen activity, particularly contractions of the rumen can be seen as “ripples” on the graph. These contractions have a duration of only a few seconds (typically in the range of 1 to 10 seconds), such that the temporal resolution of pressure detection is of the essence.

As can be seen in the upper panel (A), underlying the pressure changes caused by ruminal activity a slow build-up in pressure inside the sensing volume can be observed until the pressure sensor is at its upper pressure range limit, where the pressure in the rumen cannot be recorded anymore (flat line due to saturation). The pressure build up can have various sources. For example it might be caused by outgassings of the battery, chemical or physical processes of some of the materials in the device, temperature variations, such that a gas pressure builds up inside the sensing volume that is not connected to actual pressure changes in the rumen and therefore does not reflect the true pressure in the rumen of the animal.

In comparison in the lower panel (B) of FIG. 4, the gas-permeable portion of the membrane of the device according to the invention allows for continuous pressure equalization between the sensing volume and the rumen, such that additional pressure components caused by the device are omitted to full extend.

Each time the rumen contracts the membrane might bent to obey to the pressure difference caused over the membrane. Due to its gas-permeability the membrane can relax in its original state as the gas permeates between the rumen and the sensing volume. A stiff membrane, that might be caused by a too large thickness will lead to a slow response time, such that time resolution of the device is affected such that the pressure changes caused by contractions in the rumen cannot be observed.

On the other hand if the membrane is too thin, e.g. thinner than 0.1 mm, the membrane is not stable enough to cope with the pressure changes and would rip apart during operation.

Surprisingly, while being prone to an elaborate manufacturing procedure with a milling device, when using PTFE membranes a membrane thickness allowing for a sufficiently high time resolution and stability towards external forces has been found, despite the unfavorable processing properties of PTFE in the claimed thickness regime.

The gas-permeability of the membrane therefore allows for a precise and more importantly long-term monitoring of the pressure in the rumen of an animal.

1. A device (1) for detecting a pressure in a mammal's stomach, particularly in a rumen of a ruminant, the device (1) comprising at least the following components:
a gas-filled sensing volume (12),
a pressure sensor (9) configured and arranged to detect an atmospheric gas pressure in the sensing volume (12),
an elastic membrane (5) liquid-tight forming a deformable wall portion of the sensing volume (12), wherein the elastic membrane (5) covers an opening of the sensing volume (12) and is configured and arranged to deform over the opening in response to a pressure difference over the elastic membrane (5), such that an atmospheric gas pressure change in the rumen is instantaneously detectable in the sensing volume (12),
characterized in that
the elastic membrane (5) comprises a gas-permeable portion allowing a pressure difference over the elastic membrane (5) to equalize, wherein the gas permeable portion of the membrane (5) has a thickness between 100 μm and 700 μm.

2. The device (1) according to claim 1, wherein the gas-permeable portion of the membrane (5) has a thickness that is larger than 300 μm and/or smaller than 500 μm.

3. The device (1) according to claim 1, wherein the device (1) is configured and adapted to record the pressure in the sensing volume (12) with a time resolution better than 10 seconds, wherein a pressure change over the elastic membrane (5) is transmitted to the sensing volume instantaneously.

4. The device (1) according to claim 1, wherein the pressure sensor (9) is a piezo-resistive pressure sensor, configured to detect an atmospheric gas pressure in the sensing volume.

5. The device (1) according to claim 1, wherein the elastic membrane (5) consists of the gas-permeable portion.

6. The device (1) according to claim 1, wherein the sensing volume (12) is enclosed liquid tight by the device (1).

7. The device (1) according to claim 1, wherein the elastic membrane (5), such as a mesh, gauze, or an elastic fabric.

8. The device (1) according to claim 1, wherein the gas-permeable portion comprises or consists of Polytetrafluoroethylene (PTFE).

9. The device (1) according to claim 1, wherein the gas permeable portion of the membrane is made by a milling process.

10. The device (1) according to claim 1, wherein the device (1) comprises a rigid casing (7, 14), wherein said casing (7) comprises the pressure sensor (9) and wherein the elastic membrane (5) is attached to the casing (7) such as to form or limit the sensing volume (12).

11. The device (1) according to claim 1, wherein the device (1) has an average density and a volume, adjusted such that that the device (1) permanently remains in a rumen of a ruminant.

12. The device (1) according to claim 1, wherein the device (1) comprises a radio wave transmission module (8) configured to wireless transmit sensor data generated by the pressure sensor (9).

13. The device (1) according to claim 1, wherein the device (1) does not comprise a valve configured to adjust a gas pressure in the sensing volume (12).

14. The device (1) according to claim 1, wherein the elastic membrane (5) covers an area of more than 0.5 cm².

15. The device (1) according to claim 1, wherein the elastic membrane (5) extends planar over the sensing volume (12).

16. The device according to claim 1, wherein gas permeable portion of the membrane is configured to equalize the pressure difference over the membrane within a time interval, wherein said time interval is in the range of one second to 1.200 seconds.

17. A method for measuring a stomach pressure in a rumen of a ruminant with a device (1) according to claim 1, the method comprising the following steps of:
Applying the device (1) to the rumen of the ruminant,
Periodically or continuously detecting an atmospheric pressure in the sensing volume (12), wherein the pressure is sampled with a time resolution better than ten seconds wherein a time resolution of the device for detecting a pressure change is better than or equal to 1 second,
Storing the detected pressure and/or the detected pressure change on a data storage (11) of the device (1) and/or periodically or intermittently transmitting the detected pressure and/or the detected pressure change to a receiver arranged on an outside of the ruminant.

18. The method according to claim 17, wherein the method further comprises the steps of:
From the detected pressure and/or the detected pressure change determining rumen contractions,
Determining a rumen activity, particularly a rumen contraction frequency, from the rumen contractions,
Informing a person about the determined rumen activity.

19. The device according to claim 1, wherein pressure sensor (9) is configured and arranged to detect an atmospheric gas pressure only and not a partial gas pressure.
